(19) United States
(12) Patent Application Publication
(10) Pub. No.: US 2026/0125628 A1
(43) Pub. Date: May 7, 2026

(54) DEVICE AND METHOD FOR IN VITRO MANUFACTURING AND PURIFICATION OF THERAPEUTIC MRNA

(71) Applicant: Nature's Toolbox, Inc., Rio Rancho, NM (US)

(72) Inventors: Handy Yowanto, Walnut, CA (US); Nathan Duval, Santa Fe, NM (US); Juan Alberto Allende Cabrera, Santa Fe, NM (US); Eric Michael Benner, Albuquerque, NM (US); Julian Brandon-Jones, Cambridge (GB); Martin Philip Hereford Hoole, Cambridge (GB); Christopher William Rosier, Cambridge (GB); Nicholas Warrington, Cambridge (GB); Simon Ian Burnell, Cambridge (GB); Max Franz Wehebrink, Cambridge (GB)

(21) Appl. No.: 19/383,469

(22) Filed: Nov. 7, 2025

Related U.S. Application Data
(60) Provisional application No. 63/717,881, filed on Nov. 7, 2024.
(60) Provisional application No. 63/717,884, filed on Nov. 7, 2024.

(51) Int. Cl. C12M 1/40; C12M 1/00; C12M 1/34

(52) U.S. Cl. CPC C12M 21/18; C12M 29/06; C12M 41/12; C12M 41/40; C12M 47/10

(57) ABSTRACT

The invention includes a device for manufacturing mRNA by a continuous-flow recombinant process. The device comprises a housing; a cassette engaged with the housing, the cassette having a reaction chamber therein that holds an input reaction mixture having a DNA template wherein the manufacturing occurs within the cassette. The device has a thermal assembly providing a source of heat or cooling for the cassette, a pump to engage at least one tube within the cassette to control fluid flow through the cassette during manufacturing, a valve assembly to control a direction of fluid flow through the cassette, and a software application enabling a user to selectively manage operation of the device. The invention includes the cassette and the device as separate sub-combinations. The software application includes a non-transitory computer-readable medium containing computer executable instructions and a coupled computer processor or computer to execute the method of the manufacturing of mRNA.

### CROSS REFERENCE TO RELATED APPLICATIONS

This International application claims the benefit of and priority to both U.S. Provisional Application No. 63/717,881, filed Nov. 7, 2024 and U.S. Provisional Application No. 63/717,884, filed Nov. 7, 2024. The entire specifications and figures of the above-referenced applications are hereby incorporated, in their entirety by reference.

### FIELD OF THE INVENTION

The invention generally relates to device and method for in vitro manufacturing of polynucleotides. More specifically, the invention relates to a device and method for the manufacturing of mRNA for use in therapeutic applications in which the device controls the manufacturing process through use of a disposable cassette assembly which is isolated from but communicates with the device so that the device does not have to be cleaned or sterilized between manufacturing runs.

### BACKGROUND OF THE INVENTION

Messenger RNA (mRNA) is the template molecule that is transcribed from cellular DNA and is translated into an amino acid sequence, i.e. a protein, at ribosomes in the cells of an organism. In order to control the expression level of the encoded proteins, mRNAs possess untranslated regions (UTRs) flanking the actual open reading frame (ORF) which contains the genetic information encoding the amino acid sequence. Such UTRs, termed 5′-UTR and the 3′-UTR, respectively, are sections of the mRNA located before the start codon and after the stop codon. Further, mRNA contains a poly(A) tail region which is a long sequence of adenine nucleotides which promotes export of mRNA from the nucleus, translation and to some extent protects the mRNA from degradation. Scientific and technological advances of the recent years have made mRNA a promising candidate for a variety of uses, including diagnostic applications, and therapeutic products, like vaccines.

Due to the increasing demands of the medical community to enable personalized medicine, many approaches have been developed for mRNA production at scale. Most current methods utilize fermentation to synthesize mRNA in culture from self-replicating DNA templates, then isolate the total RNA as raw material utilizing volatile organic solvents. These processes are costly, dangerous, produce hazardous waste streams that must be mediated, while the production rate is severely dependent on the performance of the producing strain and the ability to remove impurities from diverse tRNA, rRNA and host mRNA.

There exist a long-felt need for an effective in vitro mRNA manufacturing process that does not require volatile organic solvents, produces no hazardous waste stream, and costs significantly less than its fermentation-based counterpart, while generating uniform pure mRNA fit for therapeutic applications.

There also exists a long-felt need for a manufacturing device that can produce pure mRNA at scale in which the device can be controlled with a software application enabling the device to conduct the manufacturing of pure mRNA through different types of RNA transcription processes. The term “transcription” as used herein relates to any process or method of making an RNA copies of selected gene DNA sequences. The copies, messenger RNA (mRNA), carry the corresponding gene protein information encoded in DNA.

### SUMMARY OF THE INVENTION

The invention relates to a device and method for the manufacturing of mRNA for use in therapeutic applications. The device is capable of handling all steps involved in the process of synthesizing and purifying RNA molecules. These steps may include in vitro transcription (IVT), capping, tailing, and purification. IVT may be generally described as a process that uses a DNA template, RNA polymerase, and other components to create single-stranded RNA molecules. The resulting RNA molecules are similar to natural eukaryotic mRNA and can regulate protein expression. Capping may be generally described as a process that adds a 5′ cap to an mRNA molecule. Capping can be achieved co-transcriptionally in which a cap analog is added to an IVT reaction mixture. Alternatively, capping can be achieved by using enzymes, such as guanylyltransferase and 2′-O-methyltransferase after IVT. Tailing may be generally described as a process that adds a non-template nucleotide to the 3′ end of a DNA molecule. Purification may be generally described as a process that removes residual molecules, enzymes, and unincorporated nucleotides from the RNA.

With respect to the system of the present invention for producing messenger RNA (mRNA) polynucleotides in vitro and a method of the present invention for recombinant production of messenger RNA (mRNA), this provisional application incorporates by reference the pending US Application U.S. Ser. No. 17/913,392 filed on Sep. 21, 2022, which claims priority to U.S. Provisional Application Ser. No. 63/011,133 filed on Apr. 16, 2020.

According to the invention in one general aspect, the invention provides a device and method of continuous, automated and semi-automated DNA to mRNA IVT by providing a sequence of modular unit operations within the device. Accordingly, any user of the device and method is provided the capability for controlled production of custom mRNA products.

The invention includes multiple bioprocessing modules equipped with pre-configured tubing networks or arrays and corresponding harnesses, each optimized for a different stage in a mRNA production process. Based upon the particular requirements of a user, the modules can be selected to run only the bioreactions and purification workup desired or, alternatively, modules can be serialized to provide complete end-to-end mRNA production as a continuous process.

As discussed in more detail below, the device interfaces directly with consumables used to generate the mRNA products. The device contains the hardware and software for moving and sensing fluids that used in making the products, as well as providing temperature control within the device in designated zones. The consumables can be generally described as single-use components that directly contact mRNA and defined reagents throughout the manufacturing process. Conceptually, the device and the operation thereof can be broken down into components as follows:
Component 1: A sequence of durable, general purpose bioprocessing instrument modules within the device that control device functions. The instrument modules include elements such as control systems, peristaltic pump-heads, sensors and valve actuators. Each manufacturing device may have one or more instrument modules.
Component 2: A plurality of customised, single-use cassettes that contain the consumables that are encapsulated in a closed loop flow system. The cassettes dock with the instrument modules of the device. Each cassette contains according to one embodiment includes a mix of tubes, bags of reagent, connectors, and bioreactor vessels. Each cassette has a common external interface structure that allows universal docking to the device. Cassettes will differ by internal hardware and reagents, in which each cassette can be individually and pre-configured for the desired designated biochemical unit operation.
Component 3: Pre-configured manufacturing protocols, recipes or methods that can be run by the device based on the particular biochemical unit operation. The protocols/recipes/methods can be selected by a user by manual manipulation of device controls of can be selected automatically from a list of protocols/recipes in one or more computer interfaces. The protocols/recipes may include pre-run steps and post-run steps to help guide a user.
Component 4: A host computer element including software and peripherals (e.g., a mouse and keyboard) for control of the device. The software may include a plurality of user interfaces that allow the user to selectively and incrementally create protocols/recipes/methods and the ability to execute and modify pre-configured protocols/recipes/methods.
Component 5: A reagent pack, consisting of pre-filled syringes to be loaded onto cassettes during system setup.

Because the manufacturing occurs within the cassette, which is sealed and isolated from the device, the device is immediately reusable without any cleaning or sterilization. Each cassette can be individually configured to accommodate any type of mRNA processing or manufacturing desired.

The term “cassette” as used herein means a sealed enclosure that contains at least a bioreactor and associated tubing or passageways that carry a feed solution and a reaction mixture having a DNA template enabling the execution of a continuous-flow recombinant process within the cassette.

The invention described herein includes various aspects expressed in embodiments each having separate utility and functionality. These embodiments include various combinations and sub-combinations. While the invention herein may be described with such embodiments, it shall be understood that the scope of the invention. is not specifically limited to these embodiments, and the invention should also be considered in terms of scope as being commensurate with the appended claims hereto.

According to one aspect of the invention, it may be considered a device for the manufacturing of mRNA by a continuous-flow recombinant process, the device comprising: a housing; a cassette engaged with the housing, the cassette having at least one reaction chamber therein configured to hold an input reaction mixture having a DNA template, at least one continuous-flow conduit configured hold and circulate a feed solution and to further configured to be in fluid communication with the reaction chamber, and a plurality of tubes within the cassette that carry the feed solution to the reaction chamber and that deliver the manufactured mRNA to a location external to the cassette; a thermal assembly mounted within the housing for providing a source of heat or cooling to the cassette; at least one pump mounted within the housing and facing the engaged cassette, wherein the pump is selectively operated to engage at least one tube of the plurality of tubes thereby controlling fluid flow through the cassette during the manufacturing; a valve assembly mounted within the housing for controlling a direction of fluid flow through the cassette; at least one electronic controller mounted in the housing and communicating with components of the device for controlling the operation of the device during use; and a software application communicating with the electronic controller enabling a user to selectively manage operation of the device for manufacturing of mRNA.

According to this first aspect of the invention, there are many optional features that can supplement this first aspect. These optional features may include one or more of the following:
(a) The device further includes at least one bubble sensor in the housing for monitoring the presence of liquid in the tubes and through the reaction chamber;
(b) The device further includes a cassette locking assembly mounted in the housing and communicating with the cassette for locking the cassette to the housing, the cassette locking assembly having a motor, a pulley communicating with the motor, a pulley, and at least one driven belt that enables drive of the pulley and actuation of an adjacent cam that engages the housing to lock the cassette to the housing;
(c) wherein the housing further includes at least one thermal port communicating with the thermal assembly to enable heated or cooled air generated from the thermal assembly to enter the engaged cassette for desired heating or cooling;
(d) wherein the housing further includes a loading plate extending from the housing that enables the cassette to be placed thereon;
(e) wherein the pump further includes at least one pump head having a pump channel wheel that is oriented to face the engaged cassette and positioned to selectively engage at least one tube of said plurality of tubes that is exposed and mounted to the cassette;
(f) wherein the valve assembly further includes a plurality of pinch valves oriented to face the engaged cassette and positioned to selectively engage at least one tube of said plurality of tubes that is exposed and mounted to the cassette to selectively contact the tube and to thereby close the flow of fluid through the tube;
(g) wherein the plurality of pinch valves are positioned to selectively engage a corresponding number of the plurality of tubes;
(h) herein the plurality of pinch valves are operated by corresponding solenoids;
(i) the device further includes at least one pressure sensor mounted in line with at least tube of said plurality of tubes for measuring fluid pressure in said tube;
(j) the device further includes a bezel assembly mounted on a side of said housing facing said cassette for visually indicating a status of the mRNA being manufactured in the cassette;
(k) The device further includes a plurality of said devices connected to one another by at least one connecting tube for sequential manufacturing of mRNA in a desired sequence, the connecting tube interconnecting adjacent cassettes engaged with corresponding devices;
(l) wherein said software application includes at least one of: instructions for stopping, starting and control of a rate of fluid flow in the plurality of tubes; instructions for opening and closing valves to shift a direction of fluid through the tubes; instructions for controlling temperature zones for the bioreactor and fluid therein; instructions for monitoring pressure and bubbles in tubes during manufacturing; instructions for interfacing the device with the cassette mechanically, to thereby secure the cassette into position for manufacturing; and instructions for visually indicating a status of the manufacturing and cassette loading; and
(m) wherein the thermal assembly further includes: at least one heater subassembly having an electric heating element therein for producing heat to warm air passing through an adjacent heated air duct; at least one cooling subassembly having an electric cooling element therein for cooling air passing through an adjacent cooling air duct; and an exhaust duct subassembly communicating with said heating and cooling subassemblies for evacuating air within said housing that may be warmed by operation of said subassemblies.

According to another aspect of the invention, it may be considered a device for the manufacturing of mRNA by a continuous-flow recombinant process, the device comprising: a housing; a cassette engaged with the housing, the cassette having at least one reaction chamber therein configured to hold an input reaction mixture having a DNA template, at least one conduit configured hold and circulate a feed solution and to further configured to be in fluid communication with the reaction chamber, and a plurality of tubes within the cassette that carry the feed solution to the reaction chamber and that deliver the manufactured mRNA to a location external to the cassette; at least one pump mounted within the housing and facing the engaged cassette, wherein the pump is selectively operated to engage at least one tube of the plurality of tubes thereby controlling fluid flow through the cassette during the manufacturing; a valve assembly mounted within the housing for controlling a direction of fluid flow through the cassette; at least one electronic controller mounted in the housing and communicating with components of the device for controlling the operation of the device during use; and a software application communicating with the electronic controller enabling a user to selectively manage operation of the device for manufacturing of mRNA.

This second aspect of the invention may also include any one of the optional features set forth above with respect to the first aspect. This second aspect may further include a thermal assembly mounted within the housing for providing a source of heat or cooling to the cassette.

According to another aspect of the invention, it may further include as a sub-combination, namely, a cassette for engagement with a housing of a device for the manufacturing of mRNA by a continuous-flow recombinant process, the cassette comprising: at least one reaction chamber therein configured to hold an input reaction mixture having a DNA template, at least one continuous-flow conduit configured hold and circulate a feed solution and to further configured to be in fluid communication with the reaction chamber, and a plurality of tubes within the cassette that carry the feed solution to the reaction chamber and that deliver the manufactured mRNA to a location external to the cassette.

According to another aspect of the invention, it may include yet another sub-combination, namely, a device for the manufacturing of mRNA by a continuous-flow recombinant process that is engaged with a separable cassette that contains at least one reaction chamber therein configured to hold an input reaction mixture having a DNA template, at least one continuous-flow conduit configured hold and circulate a feed solution and to further configured to be in fluid communication with the reaction chamber, the device comprising: a housing; a thermal assembly mounted within the housing for providing a source of heat or cooling to the cassette; at least one pump mounted within the housing and facing the engaged cassette, wherein the pump is selectively operated to engage at least one tube of the plurality of tubes thereby controlling fluid flow through the cassette during the manufacturing; a valve assembly mounted within the housing for controlling a direction of fluid flow through the cassette; at least one electronic controller mounted in the housing and communicating with components of the device for controlling the operation of the device during use; and a software application communicating with the electronic controller enabling a user to selectively manage operation of the device for manufacturing of mRNA.

According to yet another aspect of the invention, it may include a non-transitory computer-readable medium containing computer executable instructions, wherein, when executed by a computer processor, the instructions cause the computer processor to execute a method for the manufacturing of mRNA by a continuous-flow recombinant process conducted within a separable cassette engaged with a device that controls fluid flow, temperature and pressure within the cassette, said non-transitory computer-readable medium comprising: instructions to confirm engagement of the cassette with the device enabling the device to be engaged in a manner to control the fluid flow, temperature and pressure within the cassette; instructions to initiate and complete the manufacturing of the mRNA by the continuous-flow recombinant process conducted within the separable cassette; and instructions to confirm completion of the manufacturing process and collection of the mRNA externally of the cassette.

According to yet another aspect of the invention, it may be considered a non-transitory computer-readable medium containing computer executable instructions, wherein, when executed by a computer processor, the computer executable instructions cause the computer processor to execute a method for the manufacturing of mRNA by a continuous-flow recombinant process conducted within a separable cassette engaged with a manufacturing device that controls fluid flow, temperature and pressure within the cassette, said non-transitory computer-readable medium comprising: computer executable instructions to confirm engagement of the cassette with the manufacturing device enabling the device to be engaged in a manner to control the fluid flow, temperature and pressure within the cassette; computer executable instructions to initiate and complete the manufacturing of the mRNA by the continuous-flow recombinant process conducted within the separable cassette while the cassette is engaged with the manufacturing device; computer executable instructions to confirm completion of the manufacturing process and collection of the mRNA externally of the cassette; and computer executable instructions to display continuous-flow recombinant process information to a user on a user interface regarding monitored parameters of the process as it is conducted on the manufacturing device, wherein the monitored parameters displayed include at least one of a process stage status, an estimated run time, a remaining run time, a region temperature, a pressure sensor reading, and a bubble sensor reading.

The software application may include any one of or all of the following: instructions for stopping, starting and control of a rate of fluid flow in the plurality of tubes; instructions for opening and closing valves to shift a direction of fluid through the tubes; instructions for controlling temperature zones for the bioreactor and fluid therein; instructions for monitoring pressure and bubbles in tubes during manufacturing; instructions for interfacing the device with the cassette mechanically, to thereby secure the cassette into position for manufacturing; and instructions for visually indicating a status of the manufacturing and cassette loading; and instructions to generate user interfaces provided to a user on corresponding user screens that enable a user to select predetermined parameters that monitor and control the manufacturing process.

### BRIEF DESCRIPTION OF THE DRAWINGS

Aspects, features, and advantages of the present invention will be better understood from the following detailed description taken in conjunction with the accompanying figures, all of which are given by way of illustration only and should not be construed as limiting the presently illustrated and described embodiments. Further, for structural figures of the device provided herein, these figures may not be uniformly sized to scale so that a reader of this document can better view and understand the depicted structure.

FIG. 1 is a front perspective view of the device of the invention in an embodiment of the invention with a cassette loaded or engaged with the device;

FIG. 2 is a front perspective view of the device with the cassette removed;

FIG. 3 is rear perspective view of the device with the cassette removed;

FIG. 4 is another rear perspective view of the device with the cassette removed and showing a housing in a transparent manner in order to view device components located within the housing;

FIG. 5 is another front perspective view of the device with the cassette removed and showing the housing in a transparent manner in order to again view device components located within the housing;

FIG. 6 is an exploded perspective view of selected internal components of the device including a clamp motor assembly, a thermal assembly, and a first control assembly;

FIG. 7 is an exploded perspective view of a second control assembly;

FIG. 8 is an exploded perspective view of a pump housing and two 4-way pump heads that are used to control the flow of fluid through the tube network or array within a loaded cassette;

FIG. 9 is a perspective view of a cassette clamp motor;

FIG. 10 is a perspective view of valve components mounted to the device bulkhead or frame of the device;

FIG. 11 is an exploded perspective view of a thermal control subsystem and associated ducting that is used for thermal control of the device and loaded cassette;

FIG. 12 is a perspective view of a belt driven cassette locking system that is used to couple the device to a loaded cassette during operation of the device;

FIG. 13 is an exploded perspective view of a bubble sensor assembly showing a front bulkhead piece, four bubble sensors, and a rear bulkhead piece;

FIG. 14 is a front perspective view of a cassette assembly;

FIG. 15 is a front perspective view of the cassette assembly;

FIG. 16 is another front perspective view of the cassette assembly showing a front cover plate lifted to expose manual connection ports;

FIG. 17 is a rear perspective view of the cassette assembly showing the rear surface of the cassette assembly that engages the device;

FIG. 18 is an exploded perspective view of the cassette assembly showing the major components therein;

FIG. 19 is a schematic diagram of an exemplary computer processing device showing basic components of the computer processing device that can be associated with respect to control and operation of the invention;

FIG. 20 is a schematic diagram of an exemplary user interface or screen display of a home screen;

FIG. 21 is a schematic diagram of an exemplary user interface or screen display of a method explorer in which a user can select a particular method or recipe to be executed by the manufacturing and purification device of the invention;

FIG. 22 is a schematic diagram of an exemplary user interface or screen display of the method explorer in which the user has selected the desired method and then selects a load method button so that additional method data can be viewed;

FIG. 23 is a schematic diagram of an exemplary user interface or screen display in which once the desired method is loaded, data is displayed regarding consumables for the selected method and other data as shown;

FIG. 24 is a schematic diagram of an exemplary user interface or screen display showing various instrument preparation steps to be conducted based on the selected method;

FIG. 25 is a schematic diagram of an exemplary user interface or screen display showing a system check that is conducted during execution of the selected method;

FIG. 26 is a schematic diagram of an exemplary user interface or screen display showing a system check in which the device is not properly connected or set up and therefore is not ready for use;

FIG. 27 is a schematic diagram of an exemplary user interface or screen display showing that the selected method is complete and that the teardown or post-method steps can be taken to prepare the device for another method to be selected;

FIG. 28 is a schematic diagram of an exemplary user interface or screen display showing that the selected method and teardown are complete and the user may therefore select to confirm completion;

FIGS. 29-FIG. 44 are exemplary computer user interface screens that enable a user to set up various parameters for executing desired manufacturing processes or methods; and

FIG. 45 is a schematic diagram of a cassette and internal components therein that can be used to manufacture and purify mRNA according to use of the device of the invention.

The invention herein will be further described with respect to the appended figures and detailed description set forth below.

### DETAILED DESCRIPTION

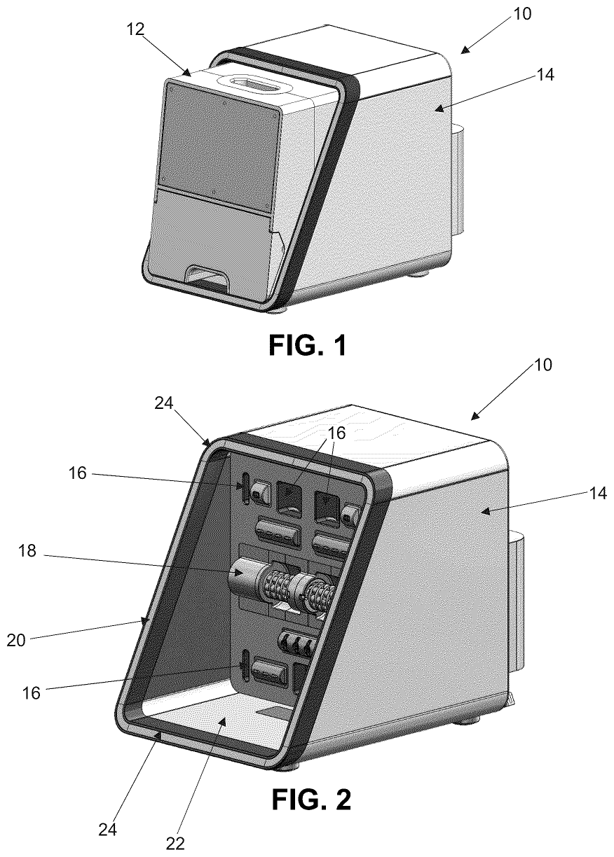

FIG. 1 is a front perspective view of the device of the invention in an embodiment of the invention with a cassette loaded or engaged with the device. Specifically, the device is generally depicted as device 10 with the outer exposed surfaces defining a housing 14. A cassette assembly 12 is shown as engaged with a large front opening on the housing.

FIG. 2 is a front perspective view of the device with the cassette removed thus exposing some additional details of the housing 14. A bulkhead plate 20 defines the surface of the device that engages the cassette assembly 12. On this surface are shown a plurality of components that are used to engage with corresponding components of the cassette assembly in order to control the manufacturing of the mRNA. A plurality of air duct openings 16 are illustrated which are used to enable a flow of air from the ducts into corresponding ducts of the cassette. The exposed portion of a pump assembly 18 is shown in which contact of portions of the pump assembly are made with tubes held within the cassette assembly. The contact facilitates the selective and incremental flow of fluid within the tubes at desired flow rates and directions. A loading plate 22 defines a lower extension of the housing which enables the cassette assembly to be vertically aligned with the bulkhead plate 20. The triangular shaped extensions of the housing 14 enable the cassette assembly to be laterally aligned with the bulkhead plate 20. A peripheral bevel assembly 24 is located on the exposed edges defining the opening to receive the cassette assembly. The bevel assembly includes a lighting capability in which the bevel assembly is illuminated in a desired color to indicate the status of the manufacturing process conducted within the cassette assembly. This external illumination assists a user in determining the status of the manufacturing without having to view a connected computer terminal or other connected peripheral.

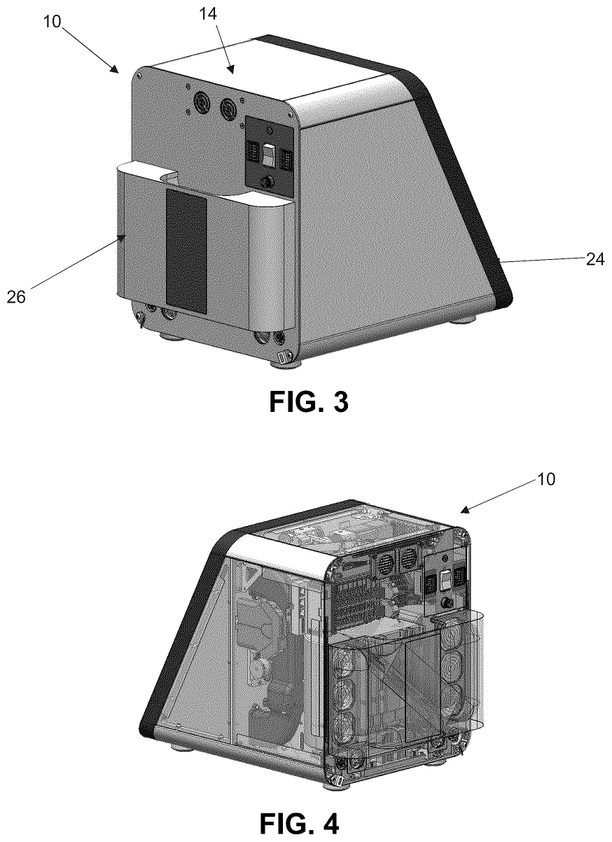

FIG. 3 is a rear perspective view of the device with the cassette removed. The rear side of the housing includes a rear panel that can be removed to access interior components of the device as desired. This figure also shows an exemplary electrical power switch and air vent openings.

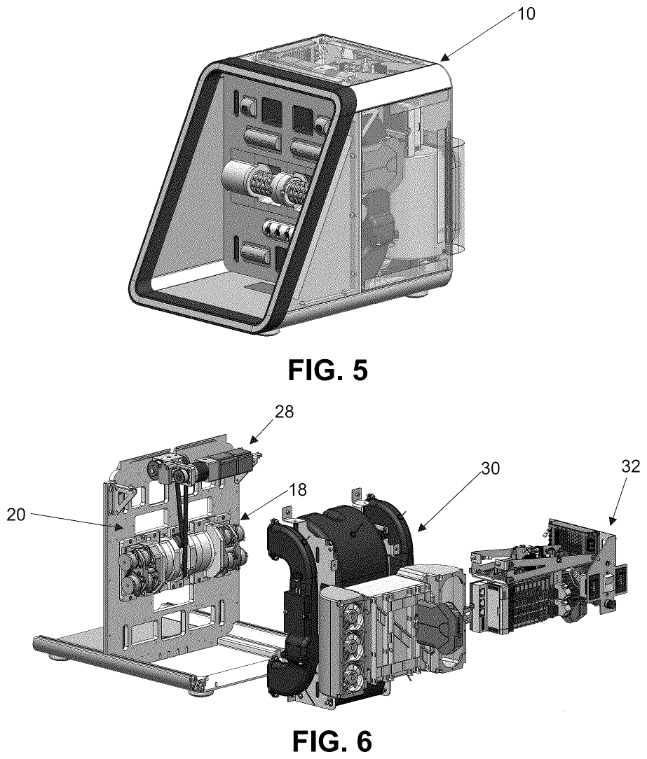

FIG. 4 is another rear perspective view of the device with the cassette removed and showing the housing in a transparent manner in order to view device components located within the housing. FIG. 5 is another front perspective view of the device with the cassette removed and again showing the housing in a transparent manner to view device components located within the housing.

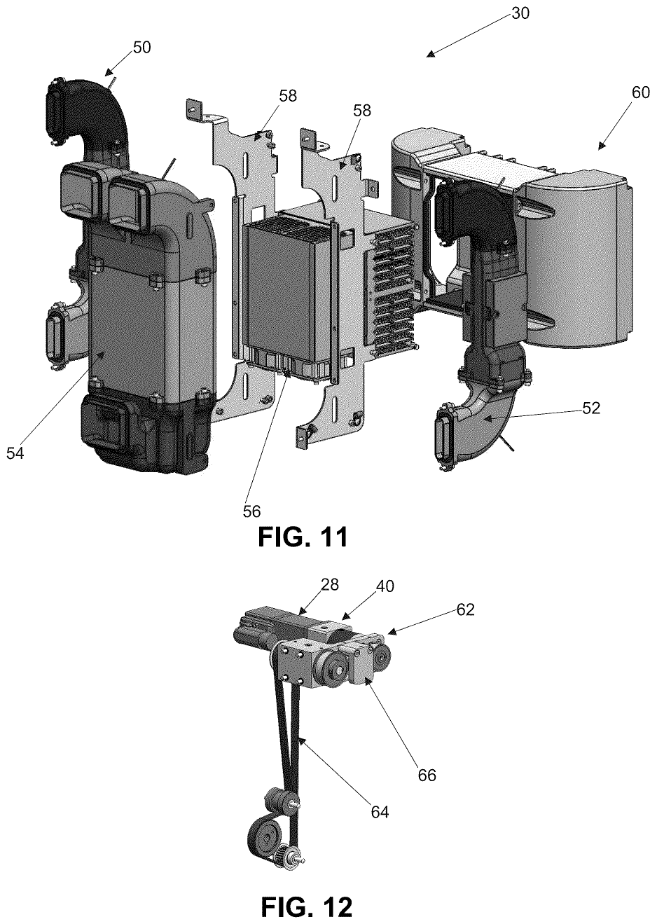

FIG. 6 is an exploded perspective view of selected internal components of the device including a clamp motor assembly 28, a thermal assembly 30, and a first control assembly 32. The clamp motor assembly 28 is used to mechanically clasp the cassette to the device. The cassette assembly must be tightly coupled to the device during operation. The clamp motor assembly is discussed in further detail with FIG. 12. The thermal assembly 30 provides heating or cooling to the cassette assembly by forced air that circulates through passages within the cassette assembly. The thermal assembly is discussed in greater detail with FIG. 11. The first control assembly 32 is the internal control element that is responsible for controlling all functions of the device except for control of the thermal assembly 30. This first control assembly may include a dedicated microprocessor and other circuitry which enables the control assembly to be programmable and to generate the necessary electronic signals for control of the various components in the device. This first control assembly also serve as a mounting chassis for the externally accessible power switch and input leads.

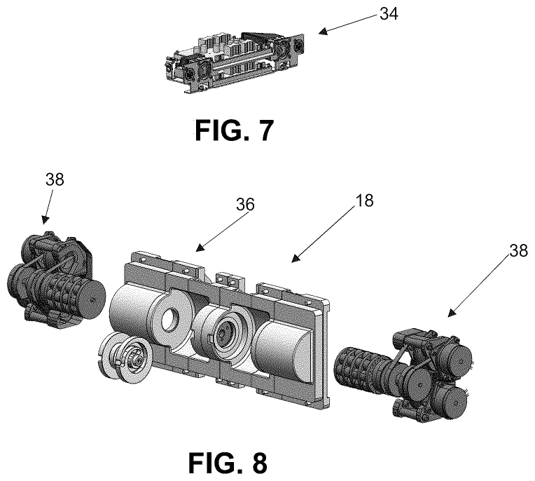

FIG. 7 is an exploded perspective view of a second control assembly. This second control assembly is specifically responsible for control of the thermal assembly. This second control assembly may also include a dedicated microprocessor and other circuitry which enables the second control assembly to be programmable and to generate the necessary electronic signals for control of the thermal assembly.

FIG. 8 is an exploded perspective view of a pump housing or bulkhead and two 4-way pump heads that are used to control the flow of fluid through the tube network or array within a loaded cassette. A bulkhead 36 houses to adjacent digital pumps that are used to selectively control fluid through tubes that carry fluid within the cassette. Each of the pumps 38 have a pump channel wheel that faces out of the device and towards the cassette, thereby allowing easy offering of the cassette tubing to the pump channel wheels.

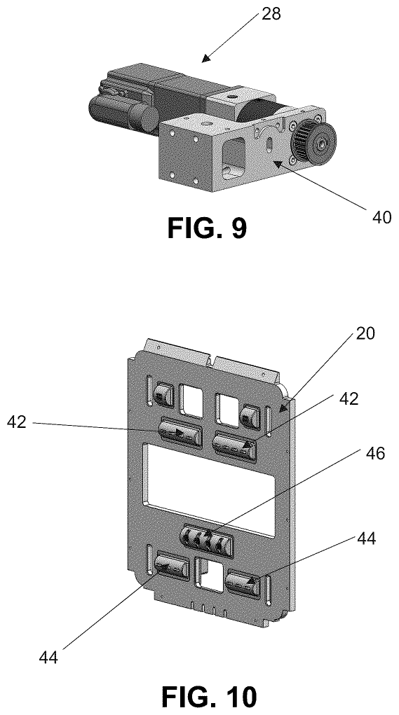

FIG. 9 is a perspective view of a cassette clamp motor 28. The clamp motor 28 is supported by a mounting frame 40 as shown. A dual cam (not shown) is mounted to a timing pulley (not shown) between the two pumps to control of the position of the cassette relative to the facing surface of the device. The cassette has corresponding pins (not shown) that engage with the cam, and together with the cassette clamp motor, they control the lateral position of the cassette.

FIG. 10 is a perspective view of valve components mounted to a valve bulkhead 42 of the device. The valve components are shown as two top valve assemblies 46 and two bottom valve assemblies 44. Collectively, these valve assemblies provide fourteen valve positions, two banks of four at the top valve assemblies and two banks of three at the bottom valve assemblies 44. As discussed below with respect to FIG. 14, valve elements of the valve assemblies are solenoid operated and mounted laterally along each of the four banks.

FIG. 11 is an exploded perspective view of a thermal control subsystem or thermal assembly 30 and associated ducting that is used for thermal control of the device and a loaded cassette. Heated air is provided to the engaged cassette by a left heater 50 and a right heater 52. Forced air passes through the heaters 50 and 52. A heating element located intermediate between the air intake and air outlet heats the passing air. Cooled air is provided by the cool air duct assembly 54 which is also shown as having an air intake and air outlet. A cooling unit 56 communicates with the cool air duct assembly 54 to provide cooling as air passes through the duct assembly 54. Mounting brackets 58 are shown for supporting the heaters and cool duct assembly. Mounted adjacent to the heating and cooling components is an exhaust duct assembly 60 that facilitates exhausting of air flow through the device since both the heaters and cooling unit generate heat during operation. The cartridge assembly is configured internally with hot and cold zones and insulating sections that insulate hot and cold zones. In both hot and cold circulation configurations within the cassette, air is pushed out to the cassette with a fan, which is monitored as it returns from circulating around media bags, bioreactors and ducting. Temperature control is managed through a thermistor inline of the airflow, which controls the level of heating and cooling.

FIG. 12 is a perspective view of a belt driven cassette locking system 62 that is used to couple the device to a loaded cassette during operation of the device; The motor 28 and belt arrangement 64 enables a cam 66 to be moved to an engaged or disengaged position.

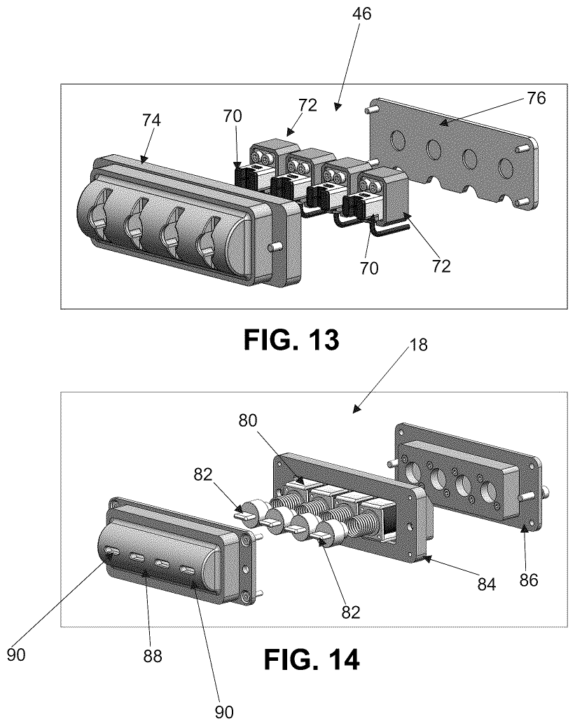

FIG. 13 is an exploded perspective view of a bubble sensor assembly showing a front bulkhead piece 74, four bubble sensors 70 with corresponding mounts 72 and a rear bulkhead piece 76. Liquid presence in the tubes is monitored by the bubble sensors mounted on the front face of the of the bulkhead plate as shown in FIG. 10. Alignment of the cassette tubing with the bulkhead bubble sensors is achieved when the cassette is in the latched position.

FIG. 14 is a front perspective view of a valve assembly 44, the valve assembly also designated as assembly 18 earlier in FIG. 2 in which only the exposed portions of the valve assembly are shown. The valve assembly includes the depicted solenoids 80 and corresponding springs. Each solenoid has a solenoid rod (not shown) that is forced forward by the spring. When a solenoid is energized, the solenoid rod is retracted. Each solenoid has a protruding anvil 82 that protrudes through the corresponding slit or aperture 90 on the front valve bulkhead 88. When a solenoid is energised, the anvil is retracted and therefore does not protrude through the corresponding slit 90. The solenoids are selectively activated to allow the anvils to protrude or retract. A protruding anvil compresses a corresponding tube on the cassette to thereby prevent fluid flow through the tube. FIG. 14 also shows the mid valve bulkhead 84 that supports the solenoids, and a rear valve bulkhead 86 that supports the rear sides of the solenoids.

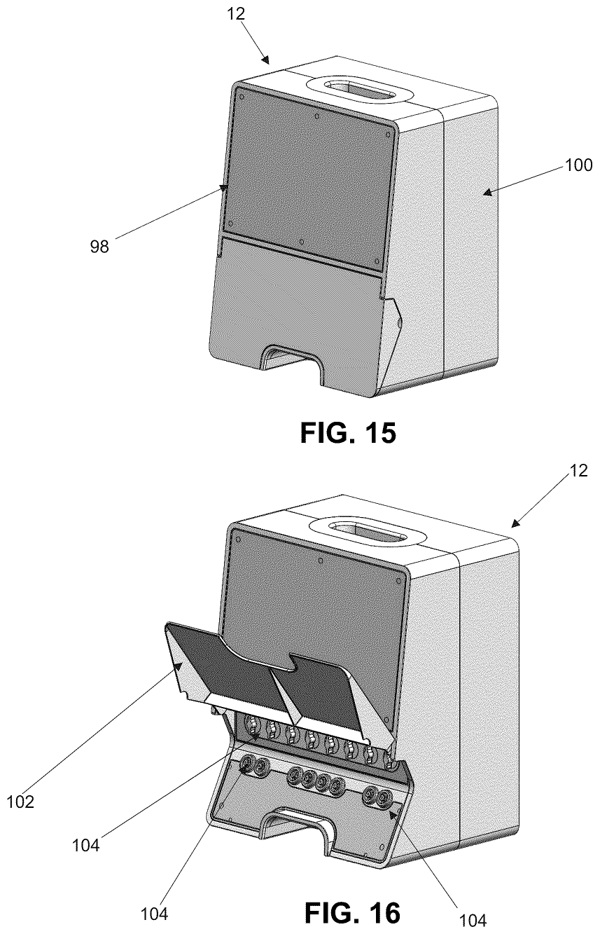

FIG. 15 is a front perspective view of the cassette assembly 12. The cassette assembly is shown generally as having a front cover 98 and a rear side 100 that faces the device when engaged therewith.

FIG. 16 is another front perspective view of the cassette assembly 12 showing a front cover plate 102 lifted to expose manual connection ports 104. Process specific reagents and fluids may be loaded into the cassette by these externally accessible connection ports. Each of these ports can be numbered and referenced in their corresponding fluidic architecture documents. Waste and product outputs are also removed via the appropriately labelled manual connection ports. Connections can be made to these ports by commercially available connectors such as male luer connectors.

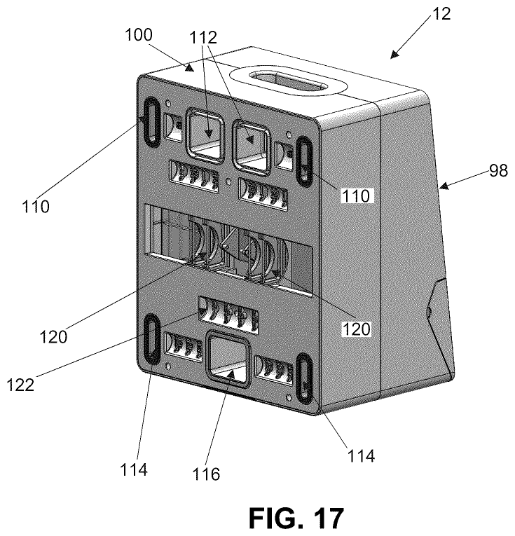

FIG. 17 is a rear perspective view of the cassette assembly showing the rear surface of the cassette assembly that engages the device. In comparing the arrangement of elements on the rear surface with the exposed elements on the facing side of the device (see FIG. 2), one will appreciate that the elements on the cassette and the device are complementary and align with one another to achieve fluid flow control through the tubes and to provide heating and cooling for the cassette. FIG. 17 more specifically shows two upper hot air inlet ports 110, two upper cold air inlet ports 112, two lower hot air outlet ports 114, and a single lower cold air outlet port 116. This figure also shows a pump interface 120 in which vertically extending tubes (not shown) are secured within the interface and are aligned with channels of the facing pump channel wheels of the device. Further, this figure also shows a bubble sensor interface 122 in which aligned vertical tubes (not shown) of the cassette are viewable through the openings in the interface 122 so that the bubble sensors can detect the presence or absence of liquid within the tubes.

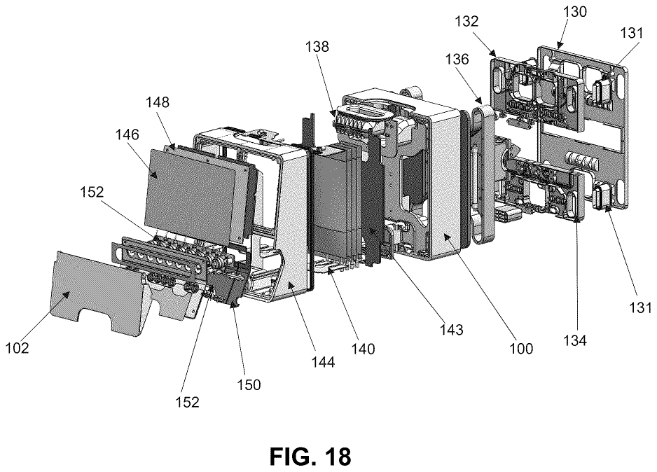

FIG. 18 is an exploded perspective view of the cassette assembly showing the major components therein. In approximate order from rear to front as this figure is presented, these components include a manual input panel 130, hot air duct connectors, an upper routing block 132, a lower routing block 134, a valve cover 136, an upper air duct assembly 138, a fluid bag support 140, an insulation panel 143, a front cover frame 144, a cover panel 146, insulation foam 148, a manual stopcock mount 150, stopcocks 152 and a manual input panel 150. As one should appreciate, the cassette assembly is a modular and fully contained, sealed unit that is capable of facilitating a desired manufacturing process.

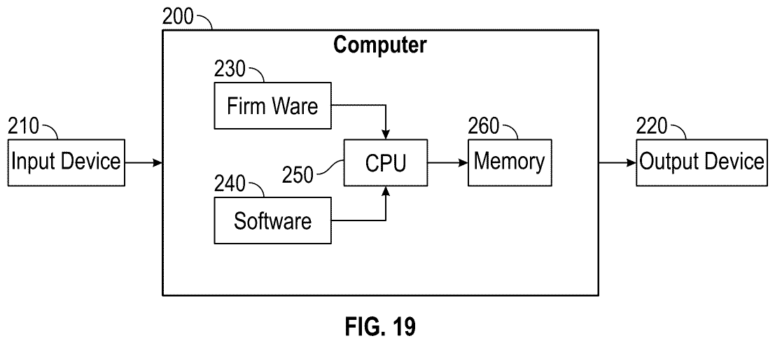

FIG. 19 is a schematic diagram of an exemplary computer processing device showing basic components of the computer processing device that can be associated with respect to control and operation of the manufacturing and purification device of the invention. As shown, the computer processing device 200 includes firmware 230, software 240, a central processing unit or CPU 250, and a memory 260. An input device 210 communicates with the processing device 200 to provide inputs, such as commands or instructions for the processing device to execute. An example of an input device includes a keyboard, mouse, or other peripheral that makes command selections for execution based on user interfaces that may be presented to the user for controlling the device of the invention. The output device 220 includes any one of various components of the device of the invention that are manipulated by commands executed from the firmware or software. Accordingly, the output device 220 could include pumps, heaters, and/or updated user interface screens that are responsive to commands generated from the firmware or software. The computer processing device 200 may also be described as a general-purpose computer that is integrated with the manufacturing device of the invention, and the general-purpose computer being further described below.

The term “software” as used herein shall be broadly interpreted to include all information processed by a computer processing device, a microcontroller, or processed by related computer executed programs communicating with the software. Software therefore includes computer programs, libraries, and related non-executable data, such as online documentation or digital media. Executable code makes up definable parts of the software and is embodied in machine language instructions readable by a corresponding data processor such as a central processing unit of the computer. The software may be written in any known programming language in which a selected programming language is translated to machine language by a compile, interpreter or assembler element of the associated computer.

The manufacturing and purification device of the invention herein may communicate electronically with one or more other user computers or controllers. Further, the device of the invention has its own controller functionality as set forth above with the first and second control assemblies that each have their own processors. The first and second control assemblies may each have or may share functionality as described with respect to the computer processor of FIG. 19. Accordingly, control of the device of the invention may be achieved with a combination of user commands executed by a user through the one or more computers and the first and second control assemblies.

The computers described herein to include a general-purpose computer, may comprise general purpose personal computers (including, merely by way of example, personal computers and/or laptop computers running various versions of Microsoft's Windows® and/or Apple® operating systems) and/or workstation computers running any of a variety of commercially available LINUX®, UNIX® or LINUX®-like operating systems. These user computers may also have any of a variety of applications, including for example, database client and/or server applications, and web browser applications. Alternatively, the user computers may be any other electronic device, such as a thin-client computer, Internet-enabled mobile telephone, and/or personal digital assistant, capable of communicating via a network and/or displaying and navigating web pages or other types of electronic documents.

The computers described herein may be further characterized as computers with elements that cooperate to achieve multiple functions normally associated with general purpose computers. For example, the hardware elements may include one or more central processing units (CPUs) for processing data. The computers may further include one or more input devices (e.g., a mouse, a keyboard, etc.); and one or more output devices (e.g., a display device, a printer, etc.). The computers may also include one or more storage devices. By way of example, storage device(s) may be disk drives, optical storage devices, solid-state storage device such as a random-access memory (“RAM”) and/or a read-only memory (“ROM”), which can be programmable, flash-updateable and/or the like.

Each of the computers described herein may include a computer-readable storage media reader; a communications peripheral (e.g., a modem, a network card (wireless or wired); working memory, which may include RAM and ROM devices. T

The computer-readable storage media reader can further be connected to a computer-readable storage medium, together (and, optionally, in combination with storage device(s)) comprehensively representing remote, local, fixed, and/or removable storage devices plus storage media for temporarily and/or more permanently containing computer-readable information.

The computers may also comprise various software elements and an operating system and/or other programmable code such as program code implementing a web service connector or components of a web service connector. It should be appreciated that alternate embodiments of a computer may have numerous variations from that described above. For example, customized hardware might also be used and/or particular elements might be implemented in hardware, software (including portable software, such as applets), or both. Further, connection to other computing devices such as network input/output devices may be employed.

It should also be appreciated that the methods described herein may be performed by hardware components or may be embodied in sequences of machine-executable instructions, which may be used to cause a machine, such as a general-purpose or special-purpose processor or logic circuits programmed with the instructions to perform the methods. These machine-executable instructions may be stored on one or more machine readable mediums, such as CD-ROMs or other type of optical disks, ROMs, RAMs, EPROMS, EEPROMs, magnetic or optical cards, flash memory, or other types of machine-readable mediums suitable for storing electronic instructions. Alternatively, the methods may be performed by a combination of hardware and software.

Considering the foregoing exemplary computer and communications network and elements described therein, In connection with one embodiment of the invention, it may also be considered a software program or software platform with computer coded or computer executable instructions that enable execution of the functionality associated with the device and methods described above. More specifically, the invention may be considered to further include a software program or software platform that enables the manufacturing of the mRNA.

With respect to software programs or platforms described herein, the software may be described as containing computer readable instructions that control one or more processors to execute computer software instructions making up one or more computer software programs. The computer readable instructions herein may be referred to, in short form, simply as “instructions for” completing a described task.

In connection with yet another embodiment of the invention, it may be considered a sub-combination including one or more user interfaces generated by the software and the manufacturing device.

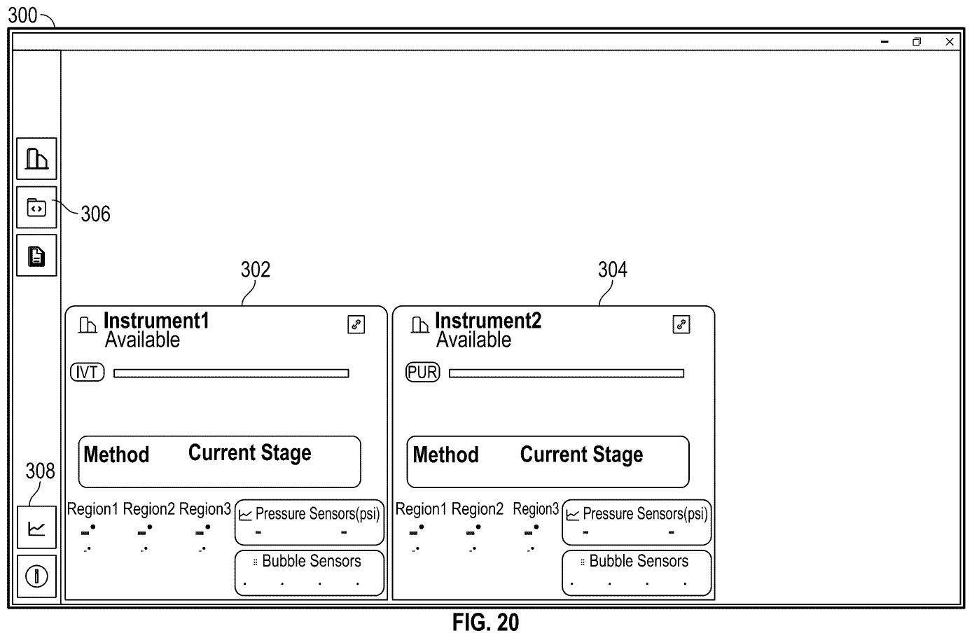

FIG. 20 is a schematic diagram of an exemplary user interface or screen display of a home screen 300. The home screen illustrates how many instruments are connected for use along with the various status indicators for the connected instruments. As shown, Instrument 1 denoted by window 302, is presented as being available for use, as well as Instrument 2, denoted by window 304, which is also presented as being available for use. Within the respective instrument windows 302 and 304 shown on this user interface, information about the instruments is shown including whether the device is set up for an IVT or purification method, the current stage of execution of the method, data regarding regions within the cassettes, and the status of pressure sensors and bubble sensors. Image or progress bars are also shown and these windows indicating what the progression is regarding the IVT or purification method. Various functions may be provided such as shown as a toolbar on the left edge of the user screen, such as a link 306 to methods or recipes that can be selected from a preexisting set of methods/recipes, and a link 308 for system maintenance.

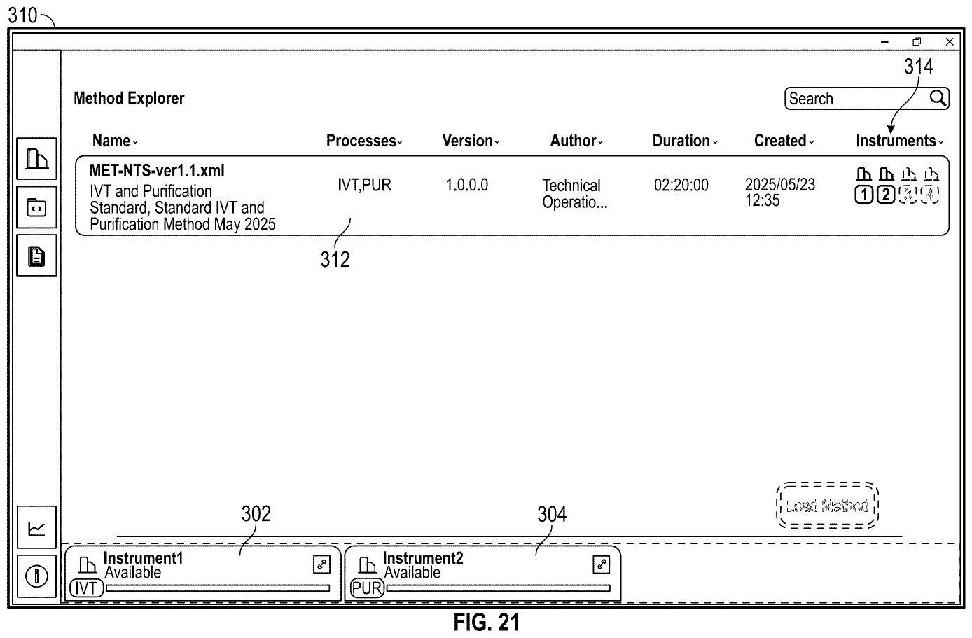

FIG. 21 is a schematic diagram of an exemplary user interface or screen display of a method explorer 310 in which a user can select a particular method or recipe to be executed by the manufacturing and purification device of the invention. A list of available methods or recipes 312 may be displayed on this screen, noting that only one method is shown in the example of FIG. 21 but it being understood that a plurality of methods can be presented on the screen. Detailed information can be listed for the recipes or methods 312, and as shown, each recipe or method may be assigned a specific name, process, version, author, duration, creation date, and instruments supported by the methods. The instruments 314 in this example shows four available instruments in which two of them are supported (1 and 2) while the other two instruments are not (3 and 4). Supported versus non-supported instruments may be distinguished by different colors or highlighting on the screen.

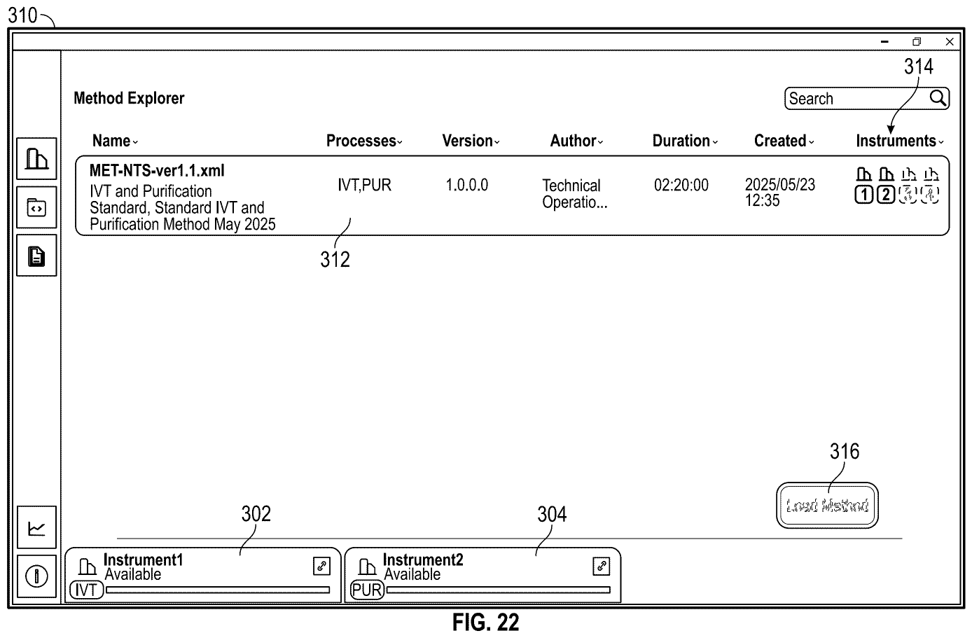

FIG. 22 is the schematic diagram of an exemplary user interface or screen display of the method explorer 310 in which the user selected the desired method and then selects a load method button 316 so that additional method data can be viewed regarding execution of the selected method.

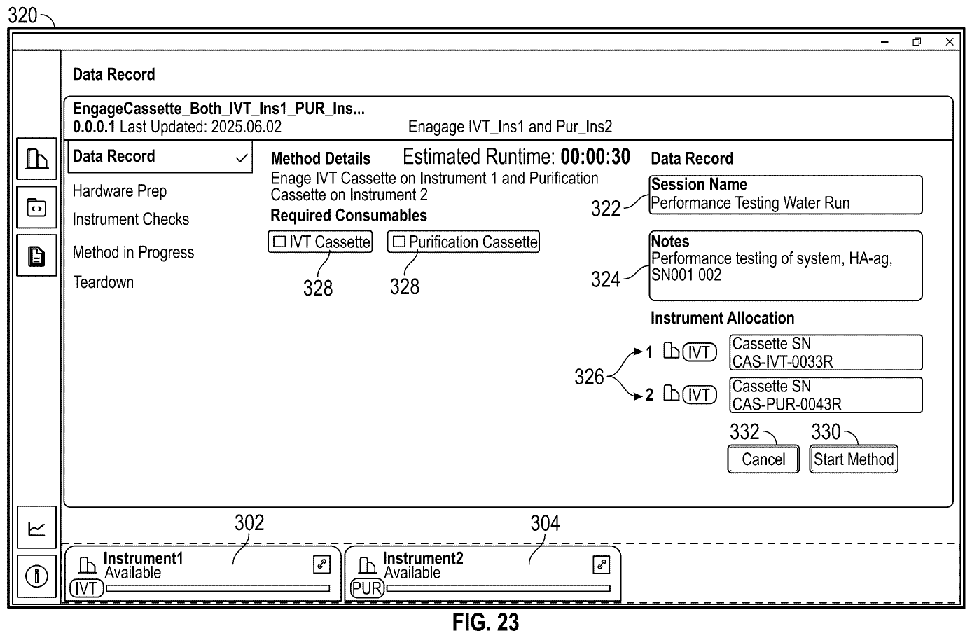

FIG. 23 is a schematic diagram of an exemplary user interface or screen display 320 in which once the desired method is loaded, data is displayed regarding consumables for the selected method and other data as shown in this figure. Among the various data fields shown in this user screen, they include a session name 322, a note field 324, and allocated instruments 326 with their respective unique cassette identifications including serial numbers. Each cassette may be individually configured to run a designated type of recipe or method. In the example shown in this figure, two cassettes are listed, each being IVT cassettes. Required consumables 328 are also listed which include in the example, an IVT cassette and a purification cassette. Instruments 1 and 2, with their corresponding windows, 302 and 304, are also shown with respective statuses—each shown as available for the selected method. If the data is shown as correct, then the user may select the start method button 330 in order to initiate the selected method. If there are errors in the data or there are other problems noted by the user, then the user may select the cancel button 332 which will prevent execution of the method and allows the user to go back and review the data and selected method to determine what changes or adjustments may be required.

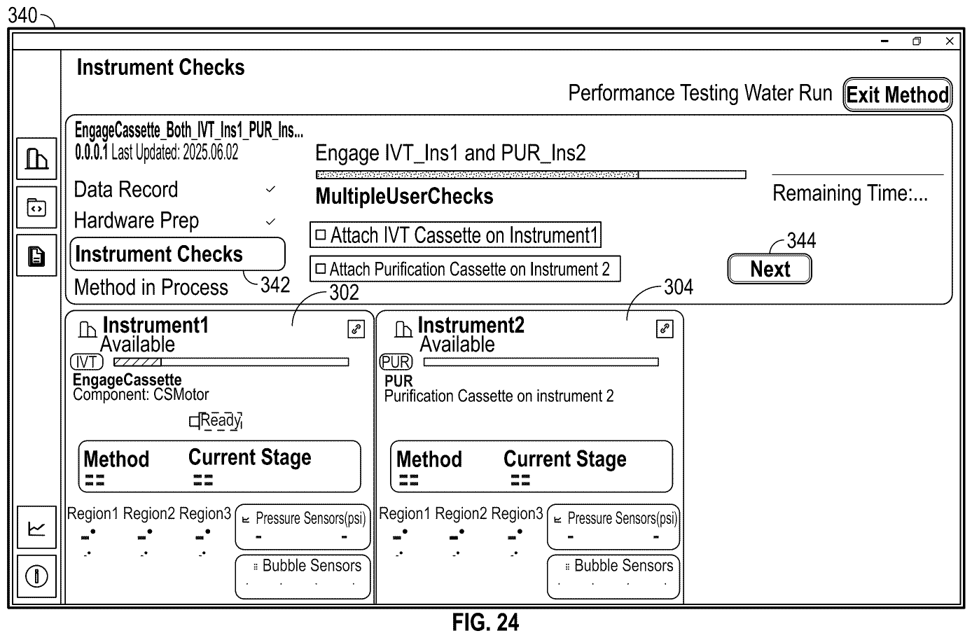

FIG. 24 is a schematic diagram of an exemplary user interface or screen display showing various instrument preparation steps to be conducted based on the selected method once the start method button 330 is selected. The instrument steps may be provided as a checklist with explanations as to how each instrument should be configured for use. In the example of this figure, simplified instrument checks 342 are shown as “Attach IVT cassette on instrument 1” and “Attach purification cassette on instrument 2”. It should be understood that other instruction checks can be listed with more detailed instrument check requirements once the instrument checks are completed, the user may then select the next button 344 which confirms instrument checks are complete.

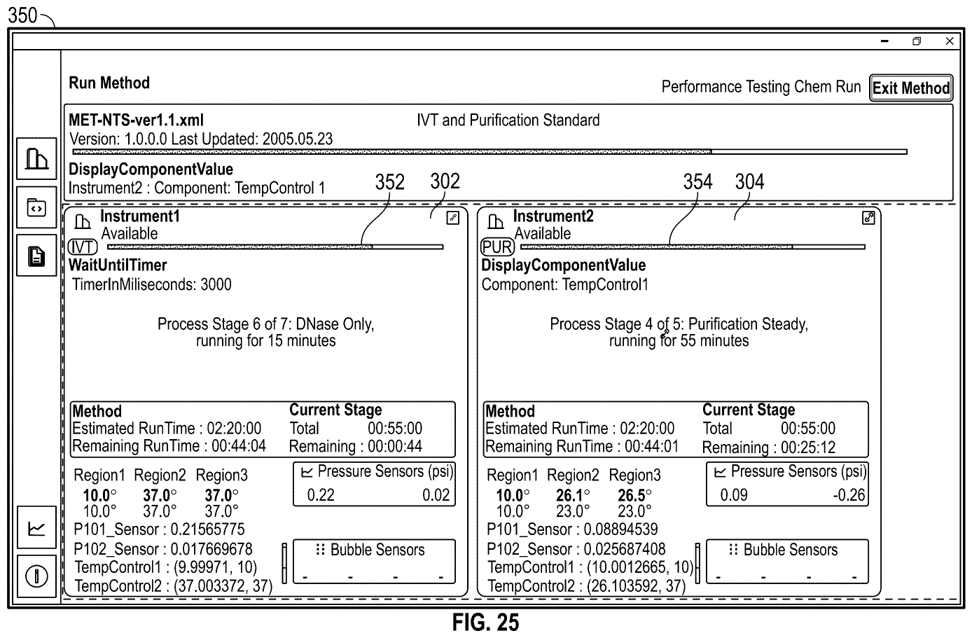

FIG. 25 is a schematic diagram of an exemplary user interface or screen display 350 showing a system check that is conducted during execution of a selected method. Each instrument window, 302 and 304 (instruments 1 and 2 as shown) provides a status of the instrument. The progress bars 352, 354, in each instrument window also provide an estimate as to the amount of completion of the selected method. Additional information shown in the instrument windows includes (1) a short description of the process stage, noting that methods can be described in terms of process steps or stages, (2) A display of the estimated run time and the remaining run time, (3) a display of regions or areas within the instruments (shown as Regions 1, 2 and 3) regarding current temperatures, (4) pressure sensor readings, (5) bubble sensor statuses, (6) and temperature control statuses. It should be appreciated that the instrument windows provided in this user interface enable a user to closely monitor the stuff a method recommended. All of the data that is displayed in these windows is automatically saved in a memory function of the manufacturing device of the invention.

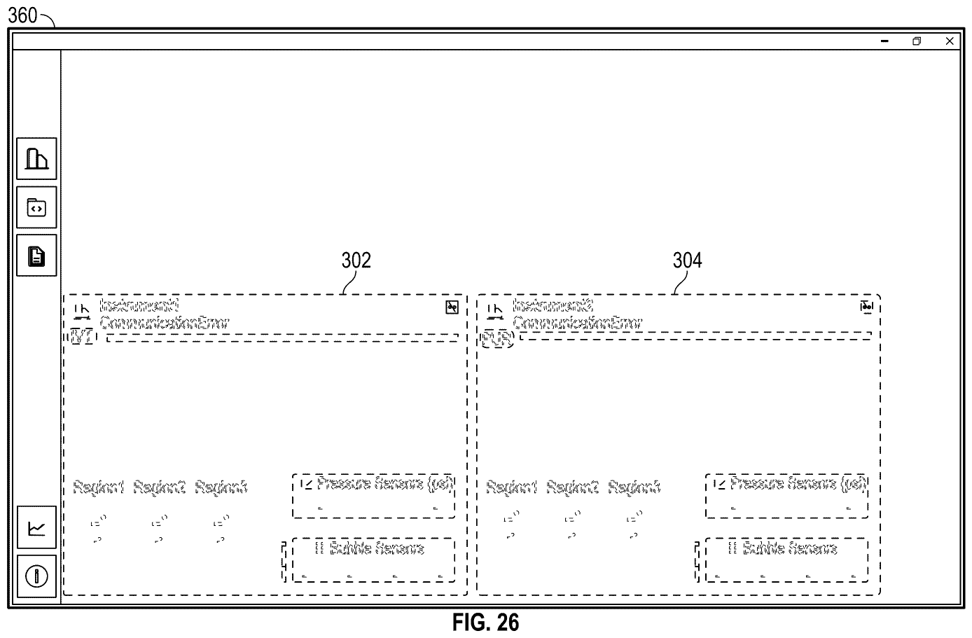

FIG. 26 is a schematic diagram of an exemplary user interface or screen display showing a system check in which the device is not properly connected or set up and therefore is not ready for use. The instrument windows 352 and 354 are shown in a lighter shade as compared to the windows in FIG. 25 to flag the user that the device is not ready for use. Other visuals can be provided to the user such as one or more error messages that specifically describe the improper connections or set up. Therefore, it should be understood that showing the instrument windows in a different shade or color is but one way in which a user can be advised of the need for addressing errors in a system check. Once the system check is complete, the manufacturing device is ready to run a method;

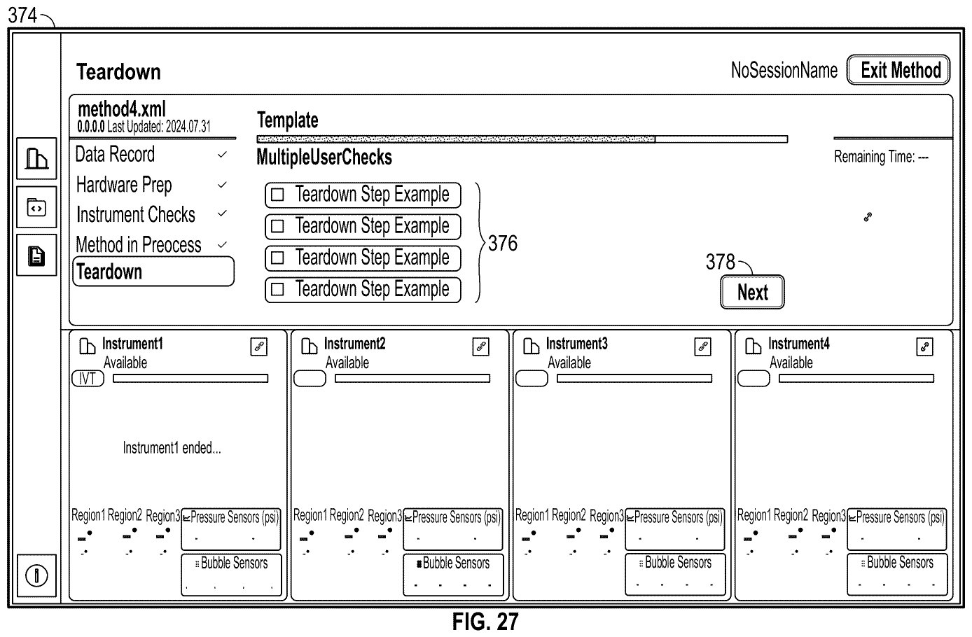

FIG. 27 is a schematic diagram of an exemplary user interface or screen display 374 showing that the selected method is complete and that teardown or post-method steps can be undertaken to prepare the device for another method to be selected. Teardown steps are listed as shown at 376 in which the user can select each teardown step to indicate that the teardown step has been completed. Once all teardown steps are completed, the user may select the “Next” button 378 to advance to the next teardown screen. Specific information on the teardown steps may be provided on this screen or another linked screen to guide the user on all actions required to prepare the device(s) for subsequent use.

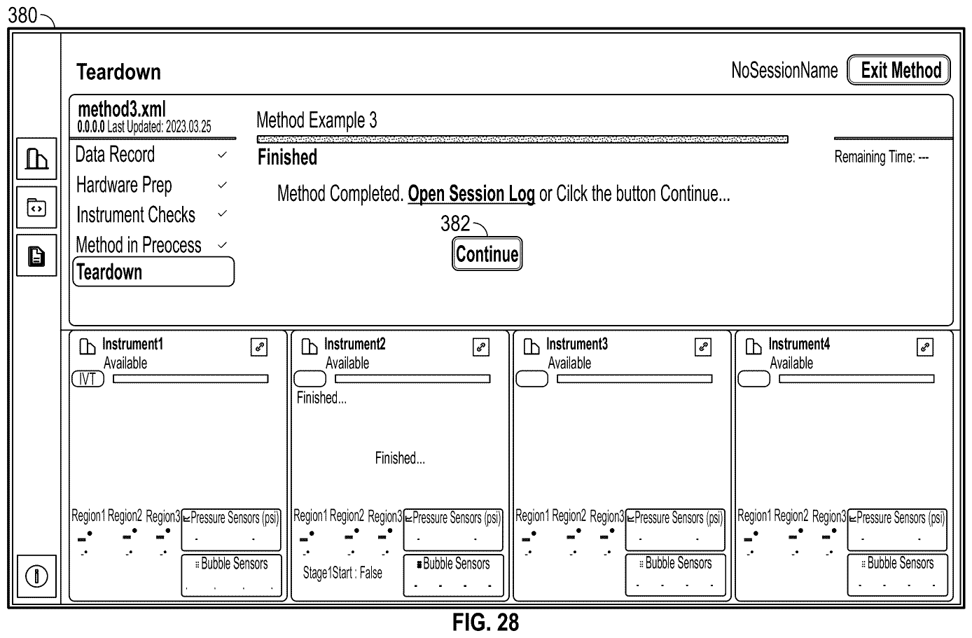

FIG. 28 is a schematic diagram of an exemplary user interface or screen display 380 showing that the selected method and teardown are complete and the user may therefore select to confirm completion by selecting the “Continue” button 382. All of the connected instruments are shown as being available meaning they are ready for a next method to be conducted.

It should be understood that the data shown in the user interfaces of FIGS. 20-28 is exemplary and should not be interpreted to limit the invention or otherwise define the parameters of any particular IVT or purification method of the invention. Accordingly, the data is provided to better understand the nature of the data that can be presented to and controlled by the user.

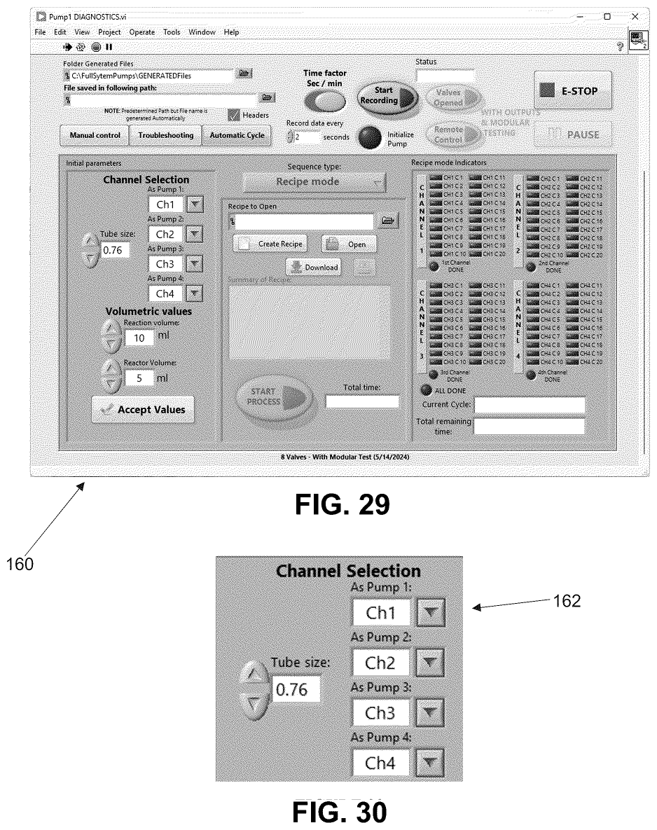

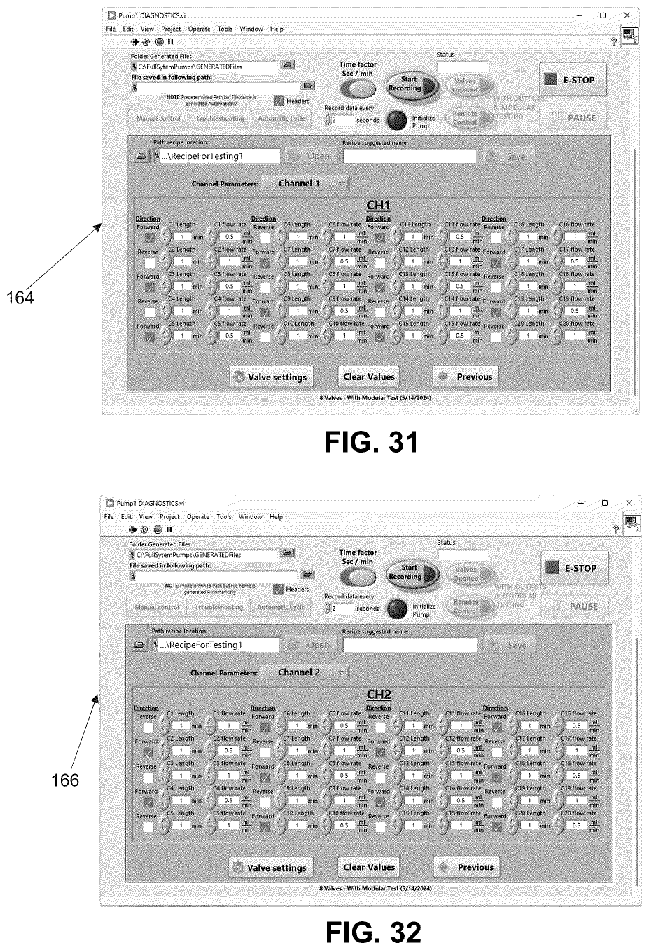

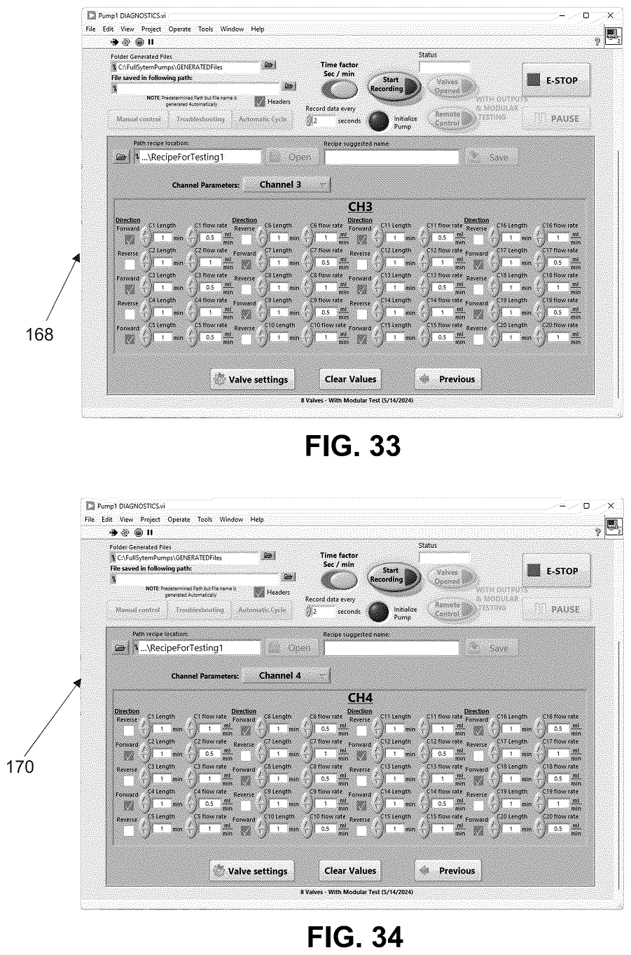

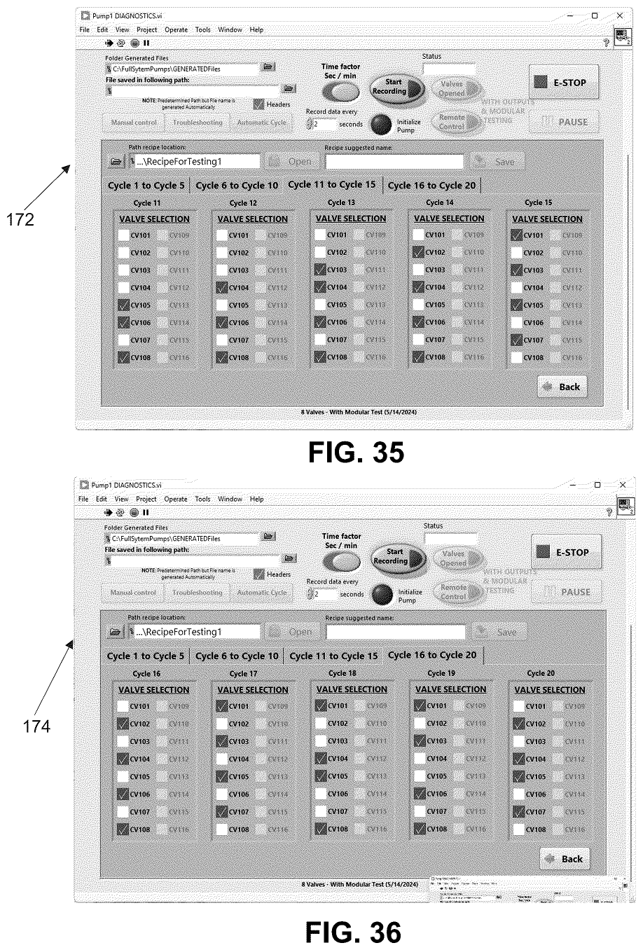

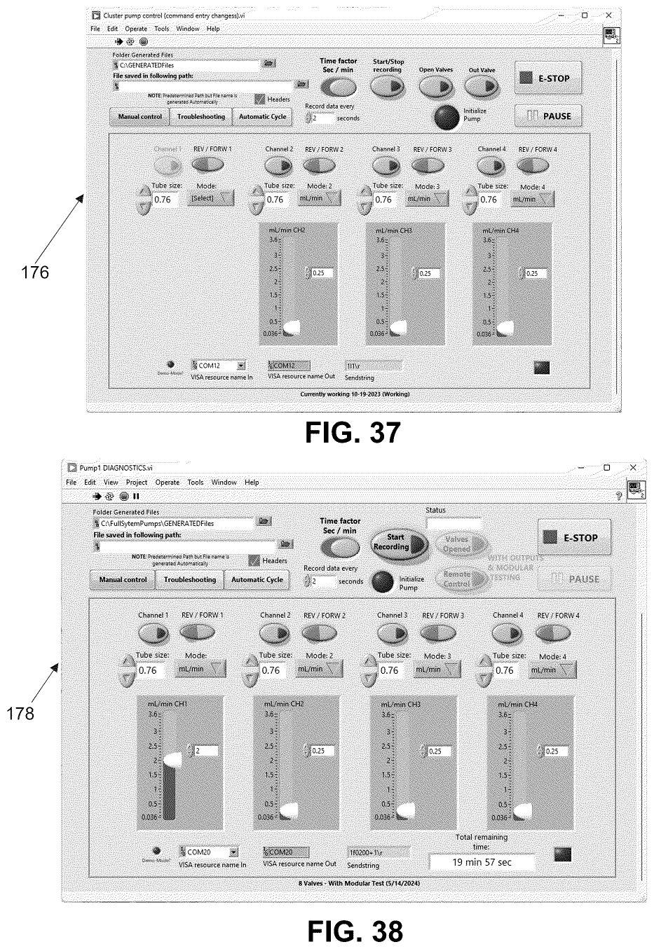

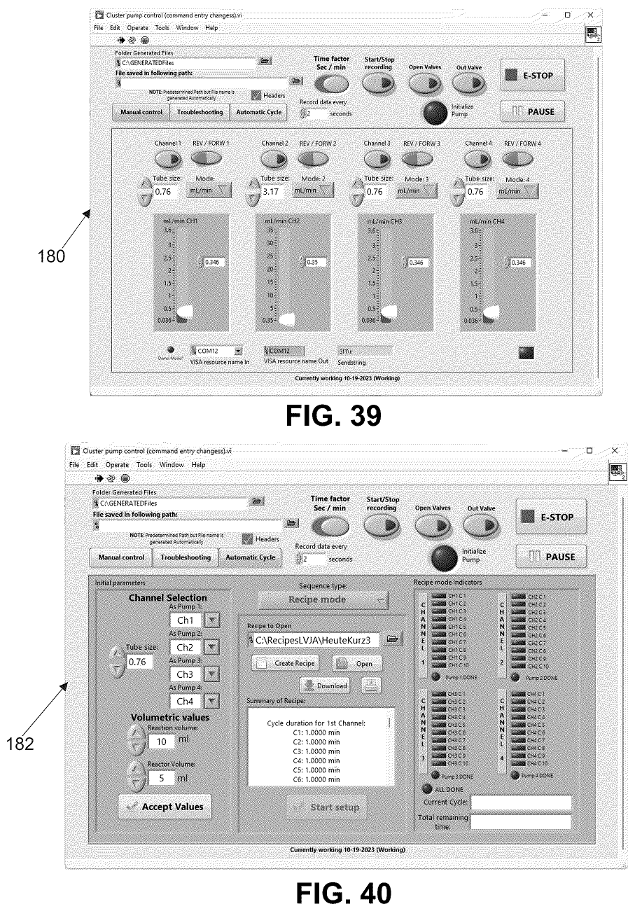

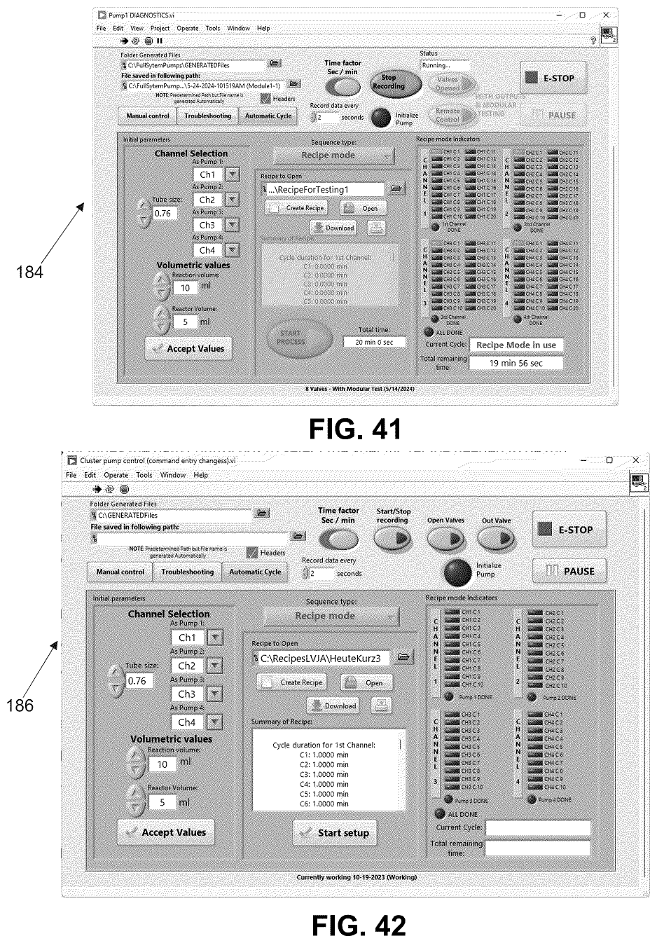

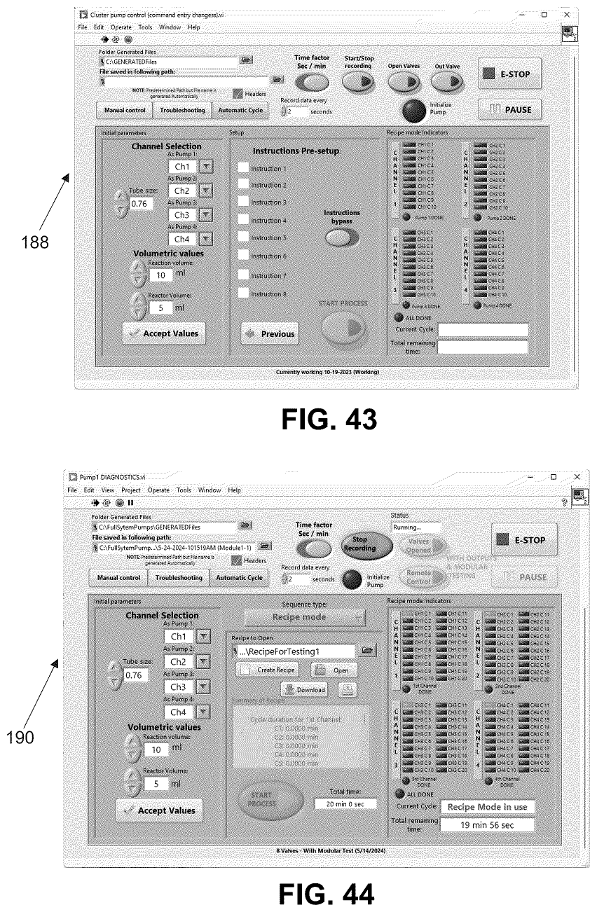

FIGS. 29-44 are exemplary computer user interface screens that enable a user to set up and control desired manufacturing processes. These user interface screens are numbered 160-190 and represent the manner in which a software application can control manufacturing processes. FIG. 30 is an enlarged portion FIG. 29 and shows that “channels” can be used as the basis for programming manufacturing sequences. These channels correspond to the channel positions on the pump assemblies in which fluid flow can be controlled through the selected tubes of a cassette. A “recipe” as displayed on the user screens corresponds to a particular manufacturing process or method that can be programmed and controlled through the user interfaces. A “cycle” as displayed on the user screens corresponds to incremental cycles or steps in a manufacturing process, and as defined by predetermined valve selections. FIGS. 37-39 show that flow rates can be incrementally monitored and controlled for a desired process, such as by a manual control protocol. A review of the other user screens will enable one to better understand the precise ability of the software application to monitor and control the manufacturing processes.

FIG. 45 is a schematic diagram of a cassette and internal components. This diagram is intended to represent any mRNA manufacturing or purification method or process that can be carried out in connection with the invention. A cassette 400 is engaged with a housing of the device of the invention. The cassette 400 has at least one reaction chamber 406 therein configured to hold an input reaction mixture having a DNA template, at least one continuous-flow conduit configured hold and circulate a feed solution 402 and to further configured to be in fluid communication with the reaction chamber. At least one tube 404 within the cassette carries the feed solution 402 to the reaction chamber 406. The manufactured mRNA 410 from the reaction chamber 406 may be delivered to a location external to the cassette as by tubing 408.

While the invention is described herein with respect to multiple preferred embodiments, it should be understood that the invention is not strictly limited to these embodiments and therefore, the invention in totality should be considered commensurate with the scope of the claims appended hereto.

What is claimed is:

1. A device for the manufacturing of mRNA by a continuous-flow recombinant process, the device comprising:
a housing;
a cassette engaged with the housing, the cassette having at least one reaction chamber therein configured to hold an input reaction mixture having a DNA template, at least one continuous-flow conduit configured hold and circulate a feed solution and to further configured to be in fluid communication with the reaction chamber, and a plurality of tubes within the cassette that carry the feed solution to the reaction chamber and that deliver the manufactured mRNA to a location external to the cassette;
a thermal assembly mounted within the housing for providing a source of heat or cooling to the cassette;
at least one pump mounted within the housing and facing the engaged cassette, wherein the pump is selectively operated to engage at least one tube of the plurality of tubes thereby controlling fluid flow through the cassette during the manufacturing; a valve assembly mounted within the housing for controlling a direction of fluid flow through the cassette;
at least one electronic controller mounted in the housing and communicating with components of the device for controlling the operation of the device during use; and
a software application communicating with the electronic controller enabling a user to selectively manage operation of the device for manufacturing of mRNA.

2. The device, as claimed in claim 1, further including:
at least one bubble sensor in the housing for monitoring the presence of liquid in the tubes and through the reaction chamber;

3. The device, as claimed in claim 1, further including:
a cassette locking assembly mounted in the housing and communicating with the cassette for locking the cassette to the housing, the cassette locking assembly having a motor, a pulley communicating with the motor, a pulley, and at least one driven belt that enables drive of the pulley and actuation of an adjacent cam that engages the housing to lock the cassette to the housing.

4. The device, as claimed in claim 1, wherein the housing further includes:
at least one thermal port communicating with the thermal assembly to enable heated or cooled air generated from the thermal assembly to enter the engaged cassette for desired heating or cooling.

5. The device, as claimed in claim 1, wherein the housing further includes:
a loading plate extending from the housing that enables the cassette to be placed thereon.

6. The device, as claimed in claim 1, wherein the pump further includes:
at least one pump head having a pump channel wheel that is oriented to face the engaged cassette and positioned to selectively engage at least one tube of said plurality of tubes that is exposed and mounted to the cassette.

7. The device, as claimed in claim 1, wherein the valve assembly further includes:
a plurality of pinch valves oriented to face the engaged cassette and positioned to selectively engage at least one tube of said plurality of tubes that is exposed and mounted to the cassette to selectively contact the tube and to thereby close the flow of fluid through the tube.

8. The device, as claimed in claim 7, wherein:
the plurality of pinch valves are positioned to selectively engage a corresponding number of the plurality of tubes.

9. The device, as claimed in claim 7, wherein:
the plurality of pinch valves are operated by corresponding solenoids.

10. The device, as claimed in claim 1, further including:
at least one pressure sensor mounted in line with at least tube of said plurality of tubes for measuring fluid pressure in said tube.

11. The device, as claimed in claim 1, further including:
a bezel assembly mounted on a side of said housing facing said cassette for visually indicating a status of the mRNA being manufactured in the cassette,

12. The device, as claimed in claim 1, further including:
a plurality of said devices connected to one another by at least one connecting tube for sequential manufacturing of mRNA in a desired sequence, the connecting tube interconnecting adjacent cassettes engaged with corresponding devices.

13. The device, as claimed in claim 1, wherein said software application includes at least one of:
computer executable instructions for stopping, starting and control of a rate of fluid flow in the plurality of tubes; computer executable instructions for opening and closing valves to shift a direction of fluid through the tubes; computer executable instructions for controlling temperature zones for the bioreactor and fluid therein; computer executable instructions for monitoring pressure and bubbles in tubes during manufacturing; computer instructions for interfacing the device with the cassette mechanically, to thereby secure the cassette into position for manufacturing; and computer instructions for visually indicating a status of the manufacturing and cassette loading.

14. The device, as claimed in claim 1, wherein the thermal assembly further includes:
at least one heater subassembly having an electric heating element therein for producing heat to warm air passing through an adjacent heated air duct;
at least one cooling subassembly having an electric cooling element therein for cooling air passing through an adjacent cooling air duct; and
an exhaust duct subassembly communicating with said heating and cooling subassemblies for evacuating air within said housing that may be warmed by operation of said subassemblies.

15. A device for the manufacturing of mRNA by a continuous-flow recombinant process, the device comprising:
a housing;
a cassette engaged with the housing, the cassette having at least one reaction chamber therein configured to hold an input reaction mixture having a DNA template, at least one conduit configured hold and circulate a feed solution and to further configured to be in fluid communication with the reaction chamber, and a plurality of tubes within the cassette that carry the feed solution to the reaction chamber and that deliver the manufactured mRNA to a location external to the cassette;
at least one pump mounted within the housing and facing the engaged cassette, wherein the pump is selectively operated to engage at least one tube of the plurality of tubes thereby controlling fluid flow through the cassette during the manufacturing;
a valve assembly mounted within the housing for controlling a direction of fluid flow through the cassette;
at least one electronic controller mounted in the housing and communicating with components of the device for controlling the operation of the device during use; and
a software application communicating with the electronic controller enabling a user to selectively manage operation of the device for manufacturing of mRNA.

16. The device of claim 14 further including:
a thermal assembly mounted within the housing for providing a source of heat or cooling to the cassette.

17. The device, as claimed in claim 14, further including:
at least one bubble sensor in the housing for monitoring the presence of liquid in the tubes and through the reaction chamber.

18. The device, as claimed in claim 14, further including:
a cassette locking assembly mounted in the housing and communicating with the cassette for locking the cassette to the housing, the cassette locking assembly having a motor, a pulley communicating with the motor, a pulley, and at least one driven belt that enables drive of the pulley and actuation of an adjacent cam that engages the housing to lock the cassette to the housing.

19. The device, as claimed in claim 14, wherein the housing further includes:
at least one thermal port communicating with the thermal assembly to enable heated or cooled air generated from the thermal assembly to enter the engaged cassette for desired heating or cooling.

20. The device, as claimed in claim 14, wherein the housing further includes:
a loading plate extending from the housing that enables the cassette to be placed thereon.

21. The device, as claimed in claim 14, wherein the pump further includes:
at least one pump head having a pump channel wheel that is oriented to face the engaged cassette and positioned to selectively engage at least one tube of said plurality of tubes that is exposed and mounted to the cassette.

22. The device, as claimed in claim 14, wherein the valve assembly further includes:
a plurality of pinch valves oriented to face the engaged cassette and positioned to selectively engage at least one tube of said plurality of tubes that is exposed and mounted to the cassette to selectively contact the tube and to thereby close the flow of fluid through the tube.

23. The device, as claimed in claim 22, wherein:
the plurality of pinch valves are positioned to selectively engage a corresponding number of the plurality of tubes.

24. The device, as claimed in claim 22, wherein:
the plurality of pinch valves are operated by corresponding solenoids.

25. The device, as claimed in claim 14, further including:
at least one pressure sensor mounted in-line with at least tube of said plurality of tubes for measuring fluid pressure in said tube.

26. The device, as claimed in claim 14, further including:
a bezel assembly mounted on a side of said housing facing said cassette for visually indicating a status of the mRNA being manufactured in the cassette,

27. The device, as claimed in claim 14, further including:
a plurality of said devices connected to one another by at least one connecting tube for sequential manufacturing of mRNA in a desired sequence, the connecting tube interconnecting adjacent cassettes engaged with corresponding devices.

28. In sub-combination, a cassette for engagement with a housing of a device for the manufacturing of mRNA by a continuous-flow recombinant process, the cassette comprising:
at least one reaction chamber therein configured to hold an input reaction mixture having a DNA template, at least one continuous-flow conduit configured hold and circulate a feed solution and to further configured to be in fluid communication with the reaction chamber, and a plurality of tubes within the cassette that carry the feed solution to the reaction chamber and that deliver the manufactured mRNA to a location external to the cassette.

29. In sub-combination, a device for the manufacturing of mRNA by a continuous-flow recombinant process that is engaged with a separable cassette that contains at least one reaction chamber therein configured to hold an input reaction mixture having a DNA template, at least one continuous-flow conduit configured hold and circulate a feed solution and to further configured to be in fluid communication with the reaction chamber, the device comprising:
a housing;
a thermal assembly mounted within the housing for providing a source of heat or cooling to the cassette;
at least one pump mounted within the housing and facing the engaged cassette, wherein the pump is selectively operated to engage at least one tube of the plurality of tubes thereby controlling fluid flow through the cassette during the manufacturing; a valve assembly mounted within the housing for controlling a direction of fluid flow through the cassette;
at least one electronic controller mounted in the housing and communicating with components of the device for controlling the operation of the device during use; and a software application communicating with the electronic controller enabling a user to selectively manage operation of the device for manufacturing of mRNA.

30. A non-transitory computer-readable medium containing computer executable instructions, wherein, when executed by a computer processor, the computer executable instructions cause the computer processor to execute a method for the manufacturing of mRNA by a continuous-flow recombinant process conducted within a separable cassette engaged with a manufacturing device that controls fluid flow, temperature and pressure within the cassette, said non-transitory computer-readable medium comprising:
computer executable instructions to confirm engagement of the cassette with the manufacturing device enabling the device to be engaged in a manner to control the fluid flow, temperature and pressure within the cassette;
computer executable instructions to initiate and complete the manufacturing of the mRNA by the continuous-flow recombinant process conducted within the separable cassette while the cassette is engaged with the manufacturing device; and
computer executable instructions to confirm completion of the manufacturing process and collection of the mRNA externally of the cassette.

31. The non-transitory computer-readable medium as claimed in claim 30 further including:
computer executable instructions to generate user interfaces provided to a user with corresponding user screens that enable a user to select predetermined parameters for controlling the method of manufacturing.

32. A non-transitory computer-readable medium containing computer executable instructions, wherein, when executed by a computer processor, the computer executable instructions cause the computer processor to execute a method for the manufacturing of mRNA by a continuous-flow recombinant process conducted within a separable cassette engaged with a manufacturing device that controls fluid flow, temperature and pressure within the cassette, said non-transitory computer-readable medium comprising:
computer executable instructions to confirm engagement of the cassette with the manufacturing device enabling the device to be engaged in a manner to control the fluid flow, temperature and pressure within the cassette;
computer executable instructions to initiate and complete the manufacturing of the mRNA by the continuous-flow recombinant process conducted within the separable cassette while the cassette is engaged with the manufacturing device;
computer executable instructions to confirm completion of the manufacturing process and collection of the mRNA externally of the cassette;
computer executable instructions to display continuous-flow recombinant process information to a user on a user interface regarding monitored parameters of the process as it is conducted on the manufacturing device, wherein the monitored parameters displayed include at least one of a process stage status, an estimated run time, a remaining run time, a region temperature, a pressure sensor reading, and a bubble sensor reading.

33. The non-transitory computer-readable medium of claim 34, further including:
computer executable instructions to display instruments that are connected or not connected for use with the process.

34. The non-transitory computer-readable medium of claim 34, further including:
computer executable instructions to display teardown steps to be conducted in order to prepare one or more instruments for subsequent use in a new process.
